(19) United States
(12) Patent Application Publication
(10) Pub. No.: US 2003/0157136 A1
(43) Pub. Date: Aug. 21, 2003

(54) Cosmetic composition forming a tackifying coating comprising a polymer with a non-silicone skeleton and reactive functional groups

(76) Inventors: Henri Samain, Bievres (FR); Isabelle Rollat-Corvol, Paris (FR); Franck Giroud, Clichy (FR); Nathalie Mougin, Paris (FR); Aude Livoreil, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/321,359

(22) Filed: Dec. 18, 2002

(30) Foreign Application Priority Data
Dec. 18, 2001 (FR) 01 16386

(51) Int. Cl. A61K 7/00

(52) U.S. Cl. 424/401

(57) ABSTRACT

Cosmetic compositions, comprising at least one polymer with a non-silicone skeleton, comprising at least two reactive chemical functional groups, that is capable of forming a tackifying coating on the hair, a cosmetic process comprising the application of the composition to the hair, and also its use for producing a tackifying coating on the hair.

Disclosed herein are cosmetic compositions, comprising at least one polymer with a non-silicone skeleton, comprising at least two reactive chemical functional groups, such compositions being capable of forming a tackifying coating on the hair. Also disclosed herein is a cosmetic process comprising the application of the composition to hair and also use of the composition for producing a tackifying coating on the hair.

To determine whether the free organic functional groups (F) of a polymer (P) constitute reactive chemical functional groups, test 1 described below is carried out:

(1) a solution or a dispersion of the polymer (P) in a cosmetically acceptable solvent chosen from water, C₁ to C₄ alcohols, esters and ketones, such as water, is prepared, this solution or dispersion having a relative polymer content ranging from 0.1% to 50% by weight;

(2) for a period ranging from 1 to 60 minutes, the solution or dispersion of polymer (P) is left to stand or is subjected to at least one of the following operations:

(i) it is stirred;

(ii) it is activated by a temperature ranging from 0° C. to 100° C.;

(iii) it is activated by a pH ranging from 1 to 13;

(iv) it is activated by at least one chemical additive (A) chosen from molecules and polymers bearing free chemical functional groups capable of reacting with at least one free organic functional group (F) of the polymer (P), wherein the chemical additive (A) can be, for example, a polymer with chemical functional groups identical to those of hair, wherein the chemical functional groups are chosen from amine, alcohol, carboxylic acid, disulphide and thiol functional groups;

(3) the solution or dispersion of polymers (P) is examined by methods known to those skilled in the art, such as by infrared or RAMAN spectrometry, in order to determine whether at least one free organic functional group (F) of the polymer (P) has given rise to the formation of covalent bonds, which may link, for example:

two atoms present in free organic functional groups (F) belonging to different polymers (P),

one atom present in the polymer (P) and one atom present in the at least one chemical additive (A);

(4) the polymer (P) is termed a “polymer comprising reactive functional groups” if the formation of covalent bond(s) is detected in point (3), and provided that such a covalent bond does not result exclusively from a hydrolysis or an oxidation of the polymer.

The at least one polymer with a non-silicone skeleton, comprising at least two reactive functional groups as disclosed herein is capable of forming covalent bonds by carrying out the test 1 described above. This characteristic distinguishes the at least one polymer disclosed herein from the majority of polymers with a non-silicone skeleton, known in the field of hair compositions, which do not react, under the conditions of the test 1, to form strong bonds, but at the very most interact with each other or with additives via bonds of hydrogen bonding or salt bonding type.

The at least one polymer disclosed herein excludes polymers comprising photoactivatable reactive functional groups, i.e., polymers comprising chemical functional groups, which, when irradiated at a wavelength ranging from 200 to 800 nm, give rise, in at least one step, to the formation of new covalent bonds.

As disclosed herein, the expression “polymer with a non-silicone skeleton” means a polymer not exclusively consisting of —Si—O—Si— sequences in its main chain.

Cosmetic products intended for treating the hair often use polymers. They make it possible to obtain, for example, hairstyle holding effects, softness effects or sheen effects.

Some compositions using polymers can have drawbacks that may be inconvenient. For example, if, after applying a product containing polymers, a person passes his hand through his hair, some of the polymers may become deposited on his fingers during the contact. This transfer phenomenon, even if only partial, can leave an impression of dirty or sticky hair. The magnitude of this transfer may depend on the climatic conditions. Thus, for example, it is often pronounced in a humid environment.

Moreover, when sebum covers the hair, either along its length or at the root, and a cosmetic product is applied thereto, for instance a styling product, the product may not only be ineffective, but, what is more, it may make the hair even more artificially shiny and dirty.

Another drawback with the polymers commonly used in cosmetics lies in the fact that they occasionally dry out the hair, thus may cause an impairment in its feel and a degradation in the expected effect of the product, for example, the hairstyle fixing and/or hold effect. An additional drawback that may also be mentioned is the fact that the polymers applied to the hair can be very quickly removed during shampooing.

For example, often the polymers used to form a coating on the hair, such as a film, having surface adhesion (tack), can result in, for example, coarse and unpleasant feeling of the hair. In addition, this tackifying coating can be removed immediately upon washing the hair, and it thus may be necessary to reapply the product, at least after each shampoo wash.

There is therefore a need to produce cosmetic compositions that are improved with respect to the compositions of the prior art, and, for example, that do not transfer onto the fingers after application to the hair, do not dry out the hair, can give the hair good cosmetic properties, even in the presence of sebum, and can be remanent with respect to repeated washings.

The inventors have discovered, surprisingly and unexpectedly, that it is possible to achieve at least one of the objectives listed above by selecting the polymers introduced into tackifying cosmetic compositions according to the nature of the chemical functional groups they bear and according to the characteristics of the film that they form on the hair.

Thus disclosed herein is a cosmetic composition, such as a hair composition, comprising, in a cosmetically acceptable medium, at least one polymer with a non-silicone skeleton, comprising at least two non-photoactivatable reactive chemical functional groups, which may be identical or different, characterized in that:

(i) the composition gives, after application to keratin fibers and drying, a styling material that has a detachment profile defined by at least:

a maximum detachment force Fₘₐₓ>1 newton, and

in addition, for example, a separation energy E_{s(M/V)} of the material placed in contact with a glass surface, of less than 300 μJ,

(ii) the at least two reactive chemical functional groups are chosen from the following monovalent and divalent groups:

epoxy,

anhydride,

acid chloride,

ethyleneimino,

aldehyde,

acetal and hemiacetal,

aminal and hemiaminal,

ketone, α-halo ketone and α-hydroxy ketone,

lactone and thiolactone,

isocyanate,

thiocyanate,

N-hydroxysuccinimide ester,

imide,

imine,

imidate,

oxazoline, oxazolinium, oxazine and oxazinium,

pyridylthiol,

thiosulphate,

acetoalkylate corresponding to the formula:

—OCO—A′—COCH₃,

wherein A′ is chosen from a bond and linear and branched alkylene groups comprising from 1 to 5 carbon atoms,

AX,

ASO₂X,

wherein A is a group chosen from alkylene, arylene and aralkylene groups comprising from 1 to 22 carbon atoms, which may be optionally interrupted with at least one unsaturated ring, and may optionally comprise at least one hetero atom, such as N, S and O,

X is a leaving group chosen from halogens, OSO₃H, OSO₂CH3, OSO₂C₂H₅, OSO₂Tos, N(CH3)₃, OPO₃R₂ and CN,

wherein Tos is a tosylate group, and

R is chosen from a hydrogen atom and C₁ to C₅ alkyl radicals; and

(iii) the at least one polymer with a non-silicone skeleton, comprising at least two reactive chemical functional groups, is other than adipic acid/epoxy-propyldiethylenetriamine copolymer.

Another embodiment disclosed herein relates to a cosmetic process comprising the application of the disclosed composition.

Yet another embodiment relates to the use of the disclosed composition to produce a tackifying coating on hair.

As disclosed herein, the term “coating” means an envelope formed at the surface of each hair, after drying of the cosmetic composition. This envelope has virtually the shape of a hollow cylinder which may extend from the root to the end of the hair and which adheres strongly thereto.

Without wishing to be bound by any theory, the inventors believe that the polymers with a non-silicone skeleton present in the cosmetic compositions as disclosed herein can, on account of their identical or different reactive functional groups, react totally or partially with themselves, with each other, with the hair, which may or may not be sensitized, and/or with at least one reactive constituent of the hair composition, and may do so after application of the cosmetic composition to the hair, to form a coating. The mechanism of formation of the coating may be understood more clearly by means of the following reaction scheme examples:

1) reaction of two polymers each comprising epoxy reactive functional groups with a reactive ingredient of the composition having the formula RHN—A—NHR′,

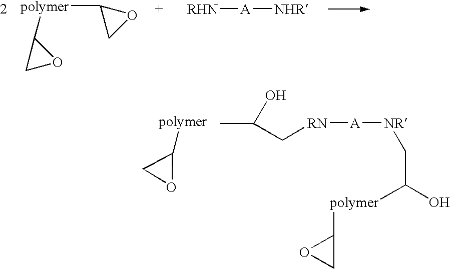

2) reaction of a polymer comprising at least two epoxy reactive functional groups with an amine function of the hair,

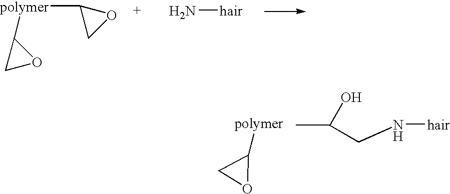

As disclosed herein, the reaction of the polymers with a non-silicone skeleton with each other and/or with the hair may, for example, be promoted by supplying heat or by adding constituents, for example, pH regulators and chemical active agents, such as oxidizing agents, reducing agents, inhibitors and polymerization catalysts.

In one embodiment, the polymer with a non-silicone skeleton, comprising at least two reactive functional groups, comprises less than 50%, in numerical terms, of carboxylic acid ester functional groups, relative to the total number of reactive chemical functional groups.

In another embodiment, the leaving group X is a halogen chosen from bromine, chlorine, iodine and fluorine.

When the at least one polymer with a non-silicone skeleton, comprising at least two reactive functional groups, comprises at least one epoxy group, the epoxy group is, for example, monovalent and is chosen from groups corresponding to formula (I):

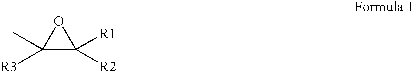

wherein R1, R2 and R3, which may be identical or different, are each chosen from:

a hydrogen atom,

linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,

aryl groups comprising from 6 to 22 carbon atoms, and

aralkyl groups, wherein the alkyl group comprises from 1 to 20 carbon atoms, and

5- to 7-membered heterocycles.

When the polymer with a non-silicone skeleton, comprising at least two reactive functional groups, comprises at least one carboxylic acid anhydride group, the carboxylic acid anhydride group is, for example, monovalent and is chosen from:

(a) groups corresponding to formula (II):

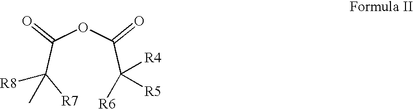

wherein R4, R5, R6, R7 and R8, which may be identical or different, have the same meanings as those given for R1, R2 and R3 in the formula (I); and

(b) groups corresponding to formula (III):

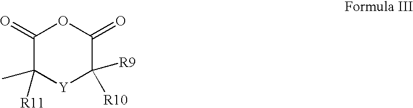

wherein Y is chosen from:

a bond,

hetero atoms chosen from O, N, S, Si and F,

alkyl and alkylene radicals that can be unsubstituted or substituted with at least one radical chosen from hydroxyl and amino radicals, comprising from 1 to 5 carbon atoms;

aralkylene radicals comprising from 7 to 10 carbon atoms, and

polydimethylsiloxane radicals comprising from 1 to 6 silicon atoms, and

wherein R9, R10 and R11, which may be identical or different, have the same meanings as those given for R1, R2 and R3 in the formula (I).

When the polymer with a non-silicone skeleton, comprising at least two reactive functional groups, comprises at least one acetoalkylate group, the acetoalkylate group is, for example, included in a group corresponding to formula (IV):

—R′₁—OCO—A′—COCH₃ Formula IV

wherein R′₁ is obtained by eliminating a hydrogen atom of the radical R1 as defined in the formula (I) and A′ has the meaning given above.

When the polymer with a non-silicone skeleton, comprising at least two reactive functional groups, comprises at least one acid chloride group, the acid chloride group is, for example, included in a group corresponding to formula (V):

—R′₁—COCl Formula V

wherein R′₁ has the same meaning as defined in the formula (IV).

When the polymer with a non-silicone skeleton, comprising at least two reactive functional groups, comprises at least one isocyanate group, the isocyanate group is, for example, included in a group corresponding to formula (VI):

—R′₁—NCO Formula VI

wherein R′₁ has the same meaning as defined in the formula (IV).

When the polymer with a non-silicone skeleton, comprising at least two reactive functional groups, comprises at least one acetal group, the acetal group is, for example, monovalent and is included in at least one of the formulae (VII), (VIII) and (IX):

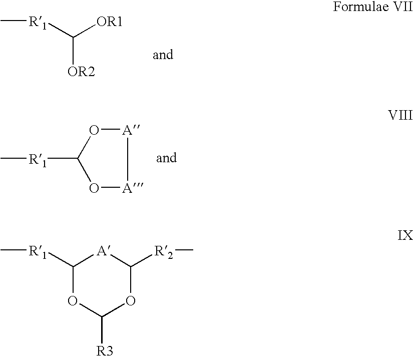

wherein:

R1, R2 and R3 have the same meaning as defined in the formula (I),

R′₁ and R′₂ are obtained by eliminating a hydrogen atom of the radicals R1 and R2 as defined in the formula (I),

A′ has the same meaning as defined above, and

A″ and A′″, which may be identical or different, are each chosen from linear and branched alkyl and alkylene groups comprising from 1 to 5 carbon atoms, which may be optionally interrupted with at least one hetero atom chosen from O, N, S, Si and F, and may be optionally substituted with at least one radical chosen from hydroxyl and amino radicals.

In one embodiment, the at least one polymer with a non-silicone skeleton comprising at least two reactive functional groups is chosen from:

(a) copolymers synthesized from (meth)acrylate and acrylate monomers, comprising acetoacetate functional groups, corresponding to the formula (IV.1):

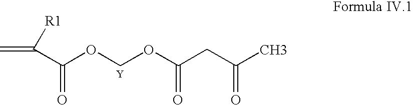

wherein R1 is chosen from H and CH₃, and Y has the same meaning as defined in the formula (III). In one embodiment, R1 is CH₃ and Y is —(CH₂)₂—,

(b) polymers synthesized from (meth)acrylate and (meth)acrylamide monomers comprising acetal functional groups, these monomers being chosen from those corresponding to the formulae (VII.1) and (VIII.1):

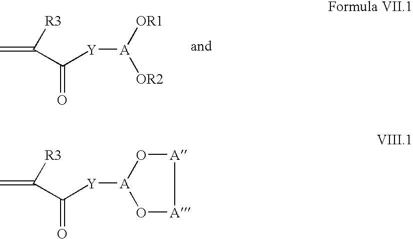

wherein Y is chosen from O and NH;

R3 is chosen from H and CH₃;

and A, A″, A′″, R1 and R2 have the same meanings as defined above.

Copolymers synthesized from N-ethyl acetal acrylamide of formula (VII.2)

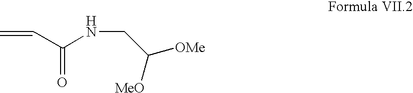

can also be used,

(c) copolymers comprising acetal functional groups, obtained by chemical modification of polymers chosen from natural and synthetic polymers, and, for example, the copolymers derived from the reaction of at least one aldehyde with poly(vinyl alcohol/vinyl acetate) of formula (X):

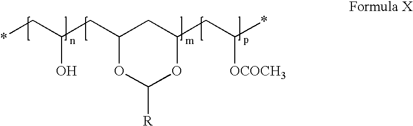

wherein R has the same meaning as defined above,

n, m and p, which may be identical or different, range from 1 to 10 000.

Such syntheses are known to those skilled in the art and are described in le Précis de Matières Plastiques, J. P. Trotigon, J. Verdu, Editions Nathan, 1996.

The polymers with a non-silicone skeleton as disclosed herein may be obtained according to the standard processes for polymerizing or modifying polymers.

To obtain such polymers, the production process may comprise, for example, at least one of the following operations:

a polycondensation,

an opening of at least one ring chosen from rings comprising from 2 to 9 carbon atoms and rings comprising from 2 to 4 silicon atoms, wherein the at least one ring may comprise at least one hetero atom, such as N, O, S and Si;

a polymerization of unsaturated monomers, chosen from free-radical and ionic polymerizations, by group transfer.

As disclosed herein, the polymer skeleton may be linear, branched, hyperbranched or dendritic. They may comprise at least one type of repeating unit, and thus may be homopolymers or copolymers which may be random, alternating or block.

As disclosed herein, the reactive functional groups are distributed along the main or side chains of the polymers, and may be optionally at the ends of the chains in the case of branched, hyperbranched and dendritic polymers.

When the polymer with a non-silicone skeleton as disclosed herein is formed by a polymerization process as described above, the reactive functional groups may be present on the monomers serving as starting material for the polymerization, or may be formed by reaction of monomers with one another during polymerization, or may be provided by at least one chemical operation in addition to the polymerization, for example, an operation comprising grafting, such as onto the polymer obtained, molecular or polymeric units bearing appropriate reactive functional groups chosen from those of formulae (I) to (IX).

To perform a polycondensation, the operating protocols described in “Step polymerization” in Principles of Polymerization, G. ODIAN, 3rd ed., Wiley Interscience, may, for example, be followed.

In the case of a polycondensation, the monomers used as the starting material are, for example, chosen from diamines and diols in reaction with diisocyanates, diacids, and diesters, which lead to polyurethanes, polyamides, polyesters and aziridines and derivatives thereof, leading to polyalkyleneimines, such as polyethyleneimines and derivatives thereof.

For example, a polyurethane may be obtained by reacting the following monomers: isophorone diisocyanate, hexamethylene diisocyanate, methylenebis-(cyclohexane diisocyanate), and polytetramethylene glycol dihydroxyl.

To perform a ring-opening operation of at least one ring chosen from rings comprising from 2 to 9 carbon atoms and rings comprising from 2 to 4 silicon atoms, wherein the at least one ring optionally comprises at least one hetero atom chosen from N, O, S and Si, the procedures described in “Ring Opening Polymerization” in Comprehensive Polymer Science, Perg. Press, vol. 3, may, for example, be followed.

In the case of a ring opening, the monomers used as the starting material to form the polymers are, for example, chosen from cyclic esters (lactones) and cyclic amides (lactams), such as,

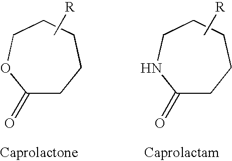

wherein R has the same meaning as defined above.

When the polymer with a non-silicone skeleton as described herein is formed by a production process comprising at least one ring-opening operation, the reactive functional groups may be present in the monomers serving as the starting material and comprising a ring, for example, as chemical substituents present on the rings, may be formed after the mutual reaction of these monomers comprising a ring, or may be provided by at least one chemical operation in addition to the ring-opening operation, for example, a separate operation comprising grafting molecular or polymeric units bearing the appropriate reactive functional groups chosen from those of formulae (I) to (IX).

To perform a free-radical or anionic polymerization, the procedures described in “Radical Polymerization and Anionic Polymerization” in Principles of Polymerization, G. ODIAN, 3rd ed., Wiley Interscience, may, for example, be followed.

In the case of a free-radical or anionic polymerization, the monomers used as starting material to form the polymers are, for example, chosen from vinyls, dienes, (meth)acrylates and (meth)acrylamides.

In the case of a free-radical or anionic polymerization, for example, the polymer may comprise at least ten units linked via covalent bonds. The reactive functional groups present on the polymer forming part of the compositions as disclosed herein, may be already present on the monomers serving as starting material for the free-radical reaction, or may possibly be formed during the free-radical reaction, or alternatively, for example, may be provided on the polymer by any additional chemical operation.

It is also possible to use natural polymers with a non-silicone skeleton and natural polymers that are chemically modified to provide them with the reactive functional groups listed above. Mention may be made, for example, and in a non-limiting manner, of polysaccharides (cellulose, chitosan, guar and derivatives thereof) and polypeptides (polyaspartic acid, polylysine and derivatives thereof). As disclosed herein, these polymers comprise, naturally or after modification, the reactive functional groups chosen from hydroxyl, amine, carboxylic acid, thiol, aldehyde and epoxy functional groups, the reactivity of which is used without further modification in the composition (for example with polymers bearing epoxy functional groups) or to provide the chemical functional groups listed above.

By way of non-limiting example, the polymer may be modified as follows:

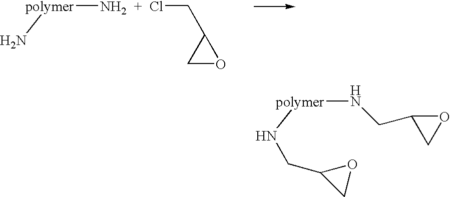

As disclosed herein, “Fₘₐₓ” means the maximum tensile force, measured using an extensometer, required to detach the respective 38 mm² surfaces of two rigid, inert, non-absorbent supports (A) and (B) placed face to face;

wherein the said surfaces are precoated with the composition at a rate of 519 μg/mm², dried for 24 hours at 22° C. under a relative humidity of 50%, and then subjected for 20 seconds to a compression of 3 newtons and finally subjected for 30 seconds to traction at a speed of 20 mm/minute.

As disclosed herein, the term “E_{s(M/V)}” means the energy supplied by the extensometer to effect the “separation” of the respective 38 mm² surfaces of two rigid, inert, non-absorbent supports (C) and (D) placed face to face; one of the supports comprising polished glass and the other of the supports being identical in nature to that of the supports (A) and (B) defined above, and the surface of which was coated with the composition at a rate of 519 μg/mm² on the support, dried for 24 hours at 22° C. under a relative humidity of 50%, and then subjected for 20 seconds to a compression of 3 newtons and finally subjected for 30 seconds to traction at a speed of 20 mm/minute.

This energy supplied by the extensometer is the work calculated using the following formula:

$${\int }_{Xs1 + 0.05}^{Xs2} F ( x ) ⅆ x$$

wherein F(x) is the force required to produce a displacement (x);

X_{S1} is the displacement (expressed in millimeters) produced by the maximum tensile force;

X_{S2} is the displacement (expressed in millimeters) produced by the tensile force allowing the total separation of the two surfaces.

Procedure Relating to the Measurement of the Fₘₐₓ

The tensile force, Fₘₐₓ, required to separate two surfaces of two rigid, inert and non-absorbent supports placed face to face and coated with the styling material to be evaluated is determined using an extensometer, for example, a machine of the Lloyd LR5K model type.

The solid, rigid, inert and non-absorbent supports may be chosen from polyethylene, polypropylene, metal alloys, and, for example, glass.

As disclosed herein, the supports used are, for example, a pair of blocks comprising, for example, a glass disc mounted on a rod required for attachment via the jaws of the extensometer. The disc is, for example, the size of the block and is fixed thereto via an adhesive such as Araldite®. The styling composition to be tested is spread as uniformly as possible over the surface of each glass disc and is made to dry such that the surface remains flat.

Discs with an area of 38 mm² are used. The amount of styling composition applied is 519 μg/mm². The drying time is 24 hours at 22° C. under a relative humidity of 50%. The rods of the two blocks are positioned in the jaws of the extensometer. The coated surfaces of the discs are then subjected to a compression phase of 3 newtons for 20 seconds by the extensometer. The traction is performed at a speed of 20 mm/minute for 30 seconds.

The detachment profile is determined by measuring the Fₘₐₓ corresponding to the maximum tensile force, measured using an extensometer, required to detach the respective surfaces of the two discs. For example, the process may be performed according to the following protocol:

Six pairs of blocks are prepared. A detachment test is performed for each pair of blocks according to the procedure as defined above. The results obtained on the six detachment profiles performed are selected, excluding for each pair of blocks the cases in which the styling materials became detached from one of the blocks of the pair. The Fₘₐₓ is determined for each remaining detachment profile. The average of these measurements is calculated.

Procedure Relating to the Measurement of the E_{s(M/V)}

The energy supplied by the extensometer to “separate” the respective surfaces of two 38 mm² rigid, inert and non-absorbent supports placed face to face is determined; one of the supports comprises polished glass and the other of the supports is identical in nature to that of the supports defined above, and the surface of which is coated and treated under the same conditions as those of the first procedure described above and using an extensometer of the same type as previously. For example, the process may be performed according to the following protocol.

Six pairs of blocks are prepared. A detachment test is performed for each pair of blocks according to the procedure described above. The results obtained on the six detachment profiles performed are selected, excluding for each pair of blocks wherein the styling materials became detached from one of the blocks of the pair. The E_{s(M/V)} is determined for each remaining detachment profile. The average of these measurements is calculated.

Another embodiment as disclosed herein is the process comprising the application to hair of the tackifying cosmetic composition.

In yet another embodiment, the process disclosed herein further comprises at least one additional operation chosen from bringing about a change in pH, an increase in temperature, adding at least one additives, and rinsing.

According to one embodiment as disclosed herein, at least one composition chosen from care, dyeing, permanent-reshaping, hair-makeup, hairstyle-fixing and hairstyle-hold compositions is applied before the application of the composition as disclosed herein.

In the compositions as disclosed herein, the at least one polymer with a non-silicone skeleton, comprising at least two reactive functional groups, is present at a concentration ranging from 0.05% to 20% by weight, for example, from 0.1% to 15% by weight, and further, for example, from 0.25% to 10% by weight, relative to the total weight of the composition.

In another embodiment disclosed herein, the composition may further comprise at least one conventional cosmetic additive chosen from fixing polymers, thickeners, anionic, nonionic, cationic and amphoteric surfactants, fragrances, preserving agents, sunscreens, proteins, vitamins, provitamins, anionic, nonionic, cationic and amphoteric non-fixing polymers, mineral, plant and synthetic oils, ceramides, pseudoceramides, linear and cyclic, modified and unmodified, volatile and non-volatile silicones, pH regulators, oxidizing agents, reducing agents, inhibitors, catalysts and any other additive conventionally used in cosmetic compositions intended to be applied to the hair.

The cosmetically acceptable medium is chosen from water, at least one cosmetically acceptable solvent, for example, alcohols, esters, ketones and cyclic volatile silicones, and water/solvent mixtures. For example, the at least one cosmetically acceptable solvent is chosen from C₁-C₄ alcohols.

When the composition as disclosed herein is packaged in an aerosol device, the composition further comprises at least one propellant, which may be chosen from volatile hydrocarbons, such as n-butane, propane, isobutane, pentane and halogenated hydrocarbons, and mixtures thereof. Carbon dioxide, nitrous oxide, dimethyl ether (DME), nitrogen or compressed air may also be used as the at least one propellant. Mixtures of propellants may also be used. For example, dimethyl ether can be used.

The at least one propellant is present, for example, in a concentration ranging from 5% to 90% by weight, and further, for example, from 10% to 60%, relative to the total weight of the composition in the aerosol device.

The compositions as disclosed herein may be applied to wet or dry hair.

The invention will be illustrated more fully with the aid of the following non-limiting example.

### EXAMPLE

1. Preparation of the Polymer P1: methyl itaconate/diethylenetriamine/epichlorohydrin/ethylenediamine Polycondensate

The procedure for manufacturing the polymer P1 comes from FR 2 252 840.

The constitution of the polymer prepared before crosslinking with epichlorohydrin may be represented by the following unit:

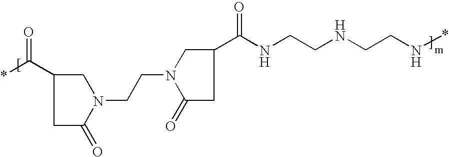

118 g (1.95 mol) of ethylenediamine were added over one hour, with stirring and under a nitrogen atmosphere, to 620 g (3.9 mol) of methyl itaconate, while keeping the temperature at 30° C.

After being left overnight at room temperature, the mixture was heated to 80° C. to remove the methanol, first at ordinary pressure and then under a reduced pressure of 15 mmHg. The appearance of a precipitate was then noted. The reaction mixture was taken up in 500 ml of benzene and the methanol-benzene azeotrope was distilled off.

The mixture was concentrated and the residue was taken up in acetone. N,N′-ethylenebis(2-methylpyrrolidone 4-carboxylate) was thus obtained, in a yield of 82%, in the form of a white powder with a melting point of 141° C. and a saponification number of 6.35 meq/g.

65.5 g (0.63 mol) of diethylenetriamine were added at room temperature to 198 g (0.63 mol) of the diester thus obtained, and the methanol formed was distilled off by heating to a temperature ranging from 120° C. to 130° C., first at ordinary pressure and then under a reduced pressure of 15 mmHg for 30 minutes.

A hard, brittle, transparent green-yellow resin that was fully water-soluble was thus obtained.

65 g of epichlorohydrin were added at room temperature to 200 g of this resin dissolved in 800 g of water. The mixture was then heated to 90° C. and an additional 10 g of epichlorohydrin were added portionwise at intervals ranging from 5 to 10 minutes. The solution was then rapidly diluted with 1135 g of water to obtain a 10% concentration.

The amount of crosslinking agent used was stoichiometric relative to the amine groups of the polyamidoamine, which ensures the presence of reactive epoxy functional groups in the molecules of the crosslinked polymer.

2. The Following Composition was Prepared:

| | | |
|---|---|---|
| Polymer P1 . . . | 5 | g |
| Monoethanolamine . . . | 1 | g |
| Water . . . | qs 100 | g |

The maximum detachment force, Fₘₐₓ, measured as indicated above, was about 2.7 N and the separation energy E_{s(M/V)} was about 240 μJ.

The composition applied to the hair and dried gave the hair a tackifying coating that was resistant to shampooing.

What is claimed is:

1. A cosmetic composition comprising, in a cosmetically acceptable medium, at least one polymer with a non-silicone skeleton, comprising at least two non-photoactivatable reactive chemical functional groups, which may be identical or different, wherein:
(i) the composition provides, after application to keratin fibers and drying, a styling material that has a detachment profile defined by at least a maximum detachment force of Fₘₐₓ>1 newton,
(ii) the at least two reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone and α-hydroxy ketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX,
ASO₂X,
wherein:
A is a group chosen from alkylene, arylene and aralkylene groups comprising from 1 to 22 carbon atoms, which may be optionally interrupted with at least one unsaturated ring, and may optionally comprise at least one hetero atom, and
X is a leaving group chosen from halogens, OSO₃H, OSO₂CH3, OSO₂C₂H₅, OSO₂Tos, N(CH3)₃, OPO₃R₂ and CN,
wherein Tos is a tosylate group, and
R is chosen from a hydrogen atom and C₁ to C₅ alkyl radicals; and
(iii) the at least one polymer with a non-silicone skeleton, comprising at least two reactive chemical functional groups, is other than adipic acid/epoxypropyldiethylenetriamine copolymer.

2. The composition according to claim 1, wherein the composition is a hair composition.

3. The composition according to claim 1 wherein, in (i), the detachment profile is further defined by a separation energy E_{s(M/V)} of the material placed in contact with a glass surface, of less than 300 μJ.

4. The composition according to claim 1, wherein the at least one hetero atom in the definition of A is chosen from N, S and O.

5. The composition according to claim 1, wherein the polymer with a non-silicone skeleton comprising at least two reactive chemical functional groups comprises less than 50%, in numerical terms, of carboxylic acid ester functional groups, relative to the total number of reactive chemical functional groups.

6. The composition according to claim 1, wherein X is a halogen chosen from bromine, chlorine, iodine and fluorine.

7. The composition according to claim 1, wherein, in (ii), the epoxy groups are monovalent and are chosen from groups corresponding to formula (I): 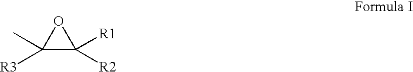
wherein R1, R2 and R3, which may be identical or different, are each chosen from:
a hydrogen atom,
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

8. The composition according to claim 1, wherein, in (ii), the anhydride group is chosen from carboxylic acid anhydride groups.

9. The composition according to claim 8, wherein the carboxylic acid anhydride groups are monovalent and are chosen from groups corresponding to formula (II): 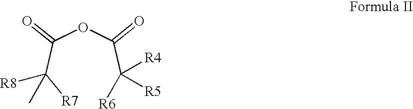
wherein R4, R5, R6, R7 and R8, which may be identical or different, are each chosen from:
a hydrogen atom,
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

10. The composition according to claim 8, wherein the carboxylic acid anhydride groups are monovalent and are chosen from groups corresponding to formula (III): 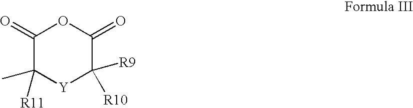
wherein Y is chosen from:
a bond,
hetero atoms chosen from O, N, S, Si and F,
alkyl and alkylene radicals that are unsubstituted or substituted with at least one radical chosen from hydroxyl and amino radicals, comprising from 1 to 5 carbon atoms,
aralkylene radicals comprising from 7 to 10 carbon atoms, and
polydimethylsiloxane radicals comprising from 1 to 6 silicon atoms, and
R9, R10 and R11, which may be identical or different, are each chosen from:
a hydrogen atom,
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

11. The composition according to claim 1, wherein, in (ii), the acetoalkylate group is included in a group corresponding to formula (IV):
—R′₁—OCO—A′—COCH₃ Formula IV
wherein R′₁ is obtained by eliminating a hydrogen atom of radical R1, wherein the radical R1 is chosen from:
a hydrogen atom,
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles; and
A′ is chosen from a bond and linear and branched alkylene groups comprising from 1 to 5 carbon atoms.

12. The composition according to claim 1, wherein, in (ii), the acid chloride group is included in a group corresponding to formula (V):
—R′₁—COCl Formula V
wherein R′₁ is obtained by eliminating a hydrogen atom of radical R1, wherein the radical R1 is chosen from:
a hydrogen atom,
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

13. The composition according to claim 1, wherein, in (ii), the isocyanate group is included in a group corresponding to formula (VI):
—R′₁—NCO Formula VI
wherein R′₁ is obtained by eliminating a hydrogen atom of radical R1, wherein the radical R1 is chosen from:
a hydrogen atom,
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles.

14. The composition according to claim 1, wherein, in (ii), the acetal group is monovalent and is included in at least one group chosen from those corresponding to formulae (VII), (VIII) and (IX): 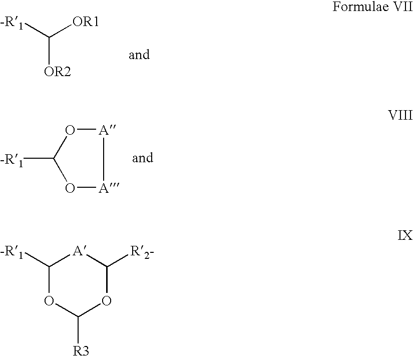
wherein:
R1, R2 and R3, which may be identical or different, are each chosen from:
a hydrogen atom,
linear and branched alkyl groups comprising from 1 to 20 carbon atoms, which may be optionally interrupted with at least one hetero atom chosen from O, N, S, Si and F, and may be optionally substituted with at least one radical chosen from hydroxyl and amino radicals,
aryl groups comprising from 6 to 22 carbon atoms,
aralkyl groups, wherein the alkyl group comprises from 1 to 20 carbon atoms, and
5- to 7-membered heterocycles;
R′₁ and R′₂ are obtained by eliminating a hydrogen atom of the radicals R1 and R2;
A′ is chosen from a bond and linear and branched alkylene groups comprising from 1 to 5 carbon atoms; and
A″ and A′″, which may be identical or different, are each chosen from linear and branched alkyl and alkylene groups comprising from 1 to 5 carbon atoms, which may be optionally interrupted with at least one hetero atom chosen from O, N, S, Si and F, and may be optionally substituted with at least one radical chosen from hydroxyl and amino radicals.

15. The composition according to claim 1, wherein the at least one polymer with a non-silicone skeleton, comprising at least two reactive chemical functional groups is obtained by a process, comprising at least one of the following operations:
a polycondensation,
an opening of at least one ring chosen from rings comprising from 2 to 9 carbon atoms and rings comprising from 2 to 4 silicon atoms, wherein the at least one ring may comprise at least one hetero atom; and
a polymerisation of unsaturated monomers, chosen from free-radical and ionic polymerizations, by group transfer.

16. The composition according to claim 15, wherein the at least one hetero atom is chosen from N, O, S and Si.

17. The composition according to claim 1, wherein the at least one polymer with a non-silicone skeleton, comprising at least two reactive chemical functional groups, is present in the composition at a concentration ranging from 0.05% to 20% by weight, relative to the total weight of the composition.

18. The composition according to claim 17, wherein the at least one polymer with a non-silicone skeleton, comprising at least two reactive chemical functional groups, is present in the composition at a concentration ranging from 0.1% to 15% by weight, relative to the total weight of the composition.

19. The composition according to claim 18, wherein the at least one polymer with a non-silicone skeleton, comprising at least two reactive chemical functional groups, is present in the composition at a concentration ranging from 0.25% to 10% by weight, relative to the total weight of the composition.

20. The composition according to claim 1, further comprising at least one cosmetic additive chosen from fixing polymers; thickeners; anionic, nonionic, cationic and amphoteric surfactants; fragrances; preserving agents; sunscreens; proteins; vitamins; provitamins; anionic, nonionic, cationic and amphoteric non-fixing polymers; mineral, plant and synthetic oils; ceramides; pseudoceramides; linear and cyclic, modified and unmodified, volatile and non-volatile silicones; pH regulators; oxidizing agents; reducing agents; inhibitors; and catalysts.

21. The composition according to claim 1, wherein the cosmetically acceptable medium is chosen from water, at least one cosmetically acceptable solvent, and mixtures thereof.

22. The composition according to claim 21, wherein the at least one cosmetically acceptable solvent is chosen from alcohols and cyclic volatile silicones.

23. The composition according to claim 22, wherein the alcohols are chosen from C₁-C₄ alcohols.

24. An aerosol device comprising at least one propellant, and a composition comprising, in a cosmetically acceptable medium, at least one polymer with a non-silicone skeleton, comprising at least two non-photoactivatable reactive chemical functional groups, which may be identical or different, wherein:
(i) the composition provides, after application to keratin fibers and drying, a styling material that has a detachment profile defined by at least a maximum detachment force Fₘₐₓ>1 newton,
(ii) the at least two reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone and α-hydroxy ketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX,
ASO₂X,
wherein:
A is a group chosen from alkylene, arylene and aralkylene groups comprising from 1 to 22 carbon atoms, which may be optionally interrupted with at least one unsaturated ring, and may optionally comprise at least one hetero atom, and
X is a leaving group chosen from halogens, OSO₃H, OSO₂CH3, OSO₂C₂H₅, OSO₂Tos, N(CH3)₃, OPO₃R₂ and CN,
wherein Tos is a tosylate group, and
R is chosen from a hydrogen atom and C₁ to C₅ alkyl radicals; and
(iii) the at least one polymer with a non-silicone skeleton, comprising at least two reactive chemical functional groups, is other than adipic acid/epoxypropyldiethylenetriamine copolymer.

25. The aerosol device according to claim 24, wherein, in (i), the detachment profile is further defined by a separation energy E_{s(M/V)} of the material placed in contact with a glass surface, of less than 300 μJ.

26. A process for cosmetic treatment of hair, comprising applying to the hair a cosmetic composition comprising, in a cosmetically acceptable medium, at least one polymer with a non-silicone skeleton, comprising at least two non-photoactivatable reactive chemical functional groups, which may be identical or different, wherein:
(i) the composition provides, after application to the hair and drying, a styling material that has a detachment profile defined by at least a maximum detachment force Fₘₐₓ>1 newton,
(ii) the at least two reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone and α-hydroxy ketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX,
ASO₂X,
wherein:
A is a group chosen from alkylene, arylene and aralkylene groups comprising from 1 to 22 carbon atoms, which may be optionally interrupted with at least one unsaturated ring, and may optionally comprise at least one hetero atom, and
X is a leaving group chosen from halogens, OSO₃H, OSO₂CH3, OSO₂C₂H₅, OSO₂Tos, N(CH3)₃, OPO₃R₂ and CN,
wherein Tos is a tosylate group, and
R is chosen from a hydrogen atom and C₁ to C₅ alkyl radicals; and
(iii) the at least one polymer with a non-silicone skeleton, comprising at least two reactive chemical functional groups, is other than adipic acid/epoxypropyldiethylenetriamine copolymer.

27. The process according to claim 26, wherein, in (i), the detachment profile is further defined by a separation energy E_{s(M/V)} of the material placed in contact with a glass surface, of less than 300 μJ.

28. The process according to claim 26, wherein before the application of the cosmetic composition, at least one composition chosen from care, dyeing, permanent-reshaping, hair-makeup, hairstyle-fixing and hairstyle-hold compositions is applied to hair.

29. A method of forming a tackifying coating on hair, comprising applying to hair a cosmetic composition comprising, in a cosmetically acceptable medium, at least one polymer with a non-silicone skeleton, comprising at least two non-photoactivatable reactive chemical functional groups, which may be identical or different, wherein:
(i) the composition provides, after application to the hair and drying, a styling material that has a detachment profile defined by at least a maximum detachment force Fₘₐₓ>1 newton,
(ii) the at least two reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone and α-hydroxy ketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX,
ASO₂X,
wherein:
A is a group chosen from alkylene, arylene and aralkylene groups comprising from 1 to 22 carbon atoms, which may be optionally interrupted with at least one unsaturated ring, and may optionally comprise at least one hetero atom, and
X is a leaving group chosen from halogens, OSO₃H, OSO₂CH3, OSO₂C₂H₅, OSO₂Tos, N(CH3)₃, OPO₃R₂ and CN,
wherein Tos is a tosylate group, and
R is chosen from a hydrogen atom and C₁ to C₅ alkyl radicals; and
(iii) the at least one polymer with a non-silicone skeleton, comprising at least two reactive chemical functional groups, is other than adipic acid/epoxypropyldiethylenetriamine copolymer.

30. The method according to claim 29, wherein, in (i), the detachment profile is further defined by a separation energy E_{s(M/V)} of the material placed in contact with a glass surface, of less than 300 μJ.

31. A composition for forming a tackifying coating on hair comprising, in a cosmetically acceptable medium, at least one polymer with a non-silicone skeleton, comprising at least two non-photoactivatable reactive chemical functional groups, which may be identical or different, wherein:
(i) the composition provides, after application to the hair and drying, a styling material that has a detachment profile defined by at least a maximum detachment force Fₘₐₓ>1 newton,
(ii) the at least two reactive chemical functional groups are chosen from the following monovalent and divalent groups:
epoxy,
anhydride,
acid chloride,
ethyleneimino,
aldehyde,
acetal and hemiacetal,
aminal and hemiaminal,
ketone, α-halo ketone and α-hydroxy ketone,
lactone and thiolactone,
isocyanate,
thiocyanate,
N-hydroxysuccinimide ester,
imide,
imine,
imidate,
oxazoline, oxazolinium, oxazine and oxazinium,
pyridylthiol,
thiosulphate,
acetoalkylate corresponding to the formula:
—OCO—A′—COCH₃,
wherein A′ is chosen from a bond and linear and branched alkylene groups comprising from 1 to 5 carbon atoms,
AX,
ASO₂X,
wherein:
A is a group chosen from alkylene, arylene and aralkylene groups comprising from 1 to 22 carbon atoms, which may be optionally interrupted with at least one unsaturated ring, and may optionally comprise at least one hetero atom, and
X is a leaving group chosen from halogens, OSO₃H, OSO₂CH3, OSO₂C₂H₅, OSO₂Tos, N(CH3)₃, OPO₃R₂ and CN,
wherein Tos is a tosylate group, and
R is chosen from a hydrogen atom and C₁ to C₅ alkyl radicals; and
(iii) the at least one polymer with a non-silicone skeleton, comprising at least two reactive chemical functional groups, is other than adipic acid/epoxypropyldiethylenetriamine copolymer,
wherein the composition is effective in forming a tackifying coating on the hair.

32. The composition according to claim 31, wherein, in (i), the detachment profile is further defined by a separation energy E_{s(M/V)} of the material placed in contact with a glass surface, of less than 300 μJ.
